SEQUENCE LISTING (8 sequences)

SEQ ID NO: 1 - DNA, 20 bp, Homo sapiens
  Human GAPDH sense specific sequence
agccgcatcttcttttgcgt

SEQ ID NO: 2 - DNA, 21 bp, Homo sapiens
  Human GAPDH antisense specific sequence
tcatatttggcaggtttttct

SEQ ID NO: 3 - DNA, 20 bp, Homo sapiens
  Human Oct-4 sense specific sequence
cgcaccactggcattgtcat

SEQ ID NO: 4 - DNA, 10 bp, Homo sapiens
  Human Oct-4 antisense specific sequence
ttctccttga

SEQ ID NO: 5 - DNA, 23 bp, Homo sapiens
  Human Nanog sense specific sequence
aatacctcagcctccagcagatg

SEQ ID NO: 6 - DNA, 23 bp, Homo sapiens
  Human Nanog antisense sequence
ctgcgtcacaccattgctattct

SEQ ID NO: 7 - DNA, 24 bp, Homo sapiens
  Human SOX2 sense specific sequence
ggcagctacgcatgatgcaggagc

SEQ ID NO: 8 - DNA, 24 bp, Homo sapiens
  Human SOX2 antisense specific sequence
ctggtcatggagttgtactgcacg

(19) United States
(12) Patent Application Publication
(10) Pub. No.: US 2014/0038289 A1
(43) Pub. Date: Feb. 6, 2014

(54) NONADHESIVE SPHERE CELL CULTURE CONTAINER, SYSTEM, AND METHOD

(71) Applicant: NATIONAL DEFENSE MEDICAL CENTER, Taipei City (TW)

(72) Inventors: Shin NIEH, Taipei City (TW); Yaoh-Shiang LIN, Taipei City (TW); Su-Feng CHEN, Taipei City (TW); Yun-Ching CHANG, Taipei City (TW); Shu-Wen JAO, Taipei City (TW)

(73) Assignee: NATIONAL DEFENSE MEDICAL CENTER, Taipei City (TW)

(21) Appl. No.: 13/714,092

(22) Filed: Dec. 13, 2012

(30) Foreign Application Priority Data
Jul. 31, 2012 (TW) 101127648

(51) Int. Cl. C12M 1/12; C12N 5/09

(52) U.S. Cl. CPC C12M 25/14; C12N 5/0693
USPC 435/397; 435/289.1

(57) ABSTRACT

The present invention provides a nonadhesive sphere cell culture container, system and method, which is performed by coating a gel film on the bottom of the culture container, and then by immersing the liquid medium containing cells on the gel film to cultivate cells as the sphere cells. Abundant cancer stem cells with sphere formation can be harvested by the invention with advantages of marked reduction of cost, time-saving (takes only 7-10 days for spheres formation), and without additional separation technique.

### CROSS-REFERENCE TO RELATED APPLICATIONS

This Non-provisional application claims priority under 35 U.S.C. §119(a) on Patent Application No(s). 101127648 filed in Taiwan, Republic of China Jul. 31, 2012, the entire contents of which are hereby incorporated by reference.

### FIELD OF THE INVENTION

This invention relates to a cell culture container and method, in particular to a culture container, system and method for nonadhesive sphere cells.

### BACKGROUND OF THE INVENTION

Cancer stem cells (CSCs) play important roles in tumor initiation, cancer progression, invasion/metastasis of cancer cells, cancer therapeutic resistance and cancer recurrence. One possible way for cancer therapy is to find out the means for cytotoxicity on CSCs. Therefore, researches involve in CSCs becomes more and more important. For obtaining CSCs for researches, there are several methods to isolate/sort CSCs.

I. Isolation based on the specific markers on cancer stem cells. The specific markers may express on the plasma membrane of some CSCs, for instance, CD133 and ALDH1 (aldehyde dehydrogenase 1) positive markers on oral cancer stem cells. These CSCs may be obtained by specifically binding these proteins with the corresponding antibodies using isolation of flow cytometry or immunomagnetic beads. However, high cost and time consumption are necessary for sorting an abundant amount of CSCs using this method.

II. Isolation with drug resistance. For example, cells which are stained with Rhodamine 123 or Hoechest 33342 fluorescent dye are sorted using flow cytometer, and thus side population cells without or with weak fluorescence signals are selected. Such isolation is made based on the principle that the expression of transport protein (e.g. MDR1 (multidrug resistance protein 1), ABCG1 (ATP-binding cassette sub-family G member 1)) within the stem cells is significantly higher than that within the normal cells, and thus the engulfed fluorescent dyes are easily discharged (Yanamoto et al., Oral Oncol. 2011, 47(9): 855-860; Song et al., PLoS One, 2010, 5(7): e11456.). Nevertheless, such isolation bears extremely low efficiency. (0.23% to 22.3% of cells only may be sorted using Hoechest 33342).

III. Isolation with chemotherapeutic agents. Cells are incubated in the culture medium supplemented with low concentration of chemotherapy drug (e.g. Cisplatin) for long time period to induce drug resistance of cells and express the characteristics of CSCs (Tsai et al., J. Oral Pathol. Med. 2011, 40(8): 621-628.). The abundantly prepared stem cells indicate that the large amounts of Cisplatin or other cancer-resistant selection drugs are essential, and the given drug resistant cells with the equivalent characteristics of CSCs may still be qualified/testified.

IV. Incubation of sphere stem cells. Cancer cells are incubated in the growth factor (e.g. epithelial growth factor (EGF), β-fibroblast growth factor (FGF-b), etc.)-rich medium, and CSCs may be isolated/sorted from the generated colonies of spheroid cells which can be enriched from a single cell in this culture medium (Chiou et al., Clin. Cancer Res. 2008, 14(13): 4085-4095; Hueng et al., J. Neurooncol. 2011, 104(1): 45-53.). However, long term incubation (4 to 6 weeks) and a plenty of laboratory resources (the considerably high cost on growth factors) are necessary for affording CSCs.

In addition, Taiwan Patent No. I360576 (i.e. Taiwan Publication No. 201009077) discloses a technology for sorting cells using the transfer plate coated with a thermosensitive and easily detachable hydrogel, wherein the transfer plate with nylon matrix is processed with plasma and co-polymerized, and then the hydrogel base material containing N-isopropylacrylamide (NIPAAm), ammonium persulfate (APS), N,N,N′,N′-tetramethyl-ethylenediamine (TEMED), N′,N′-methylenebisacrylamide (NMBA) is mixed with vitamin B₂, and NIPAAm enables surface branching polymerization using water bath, and a layer of easily detachable hydrogel is made after remove of unbound monomers and ultraviolet (UV) radiation. Subsequently, cells are cultivated in the culture medium containing EGF, FGF-b and lactoferrin until differentiating into sphere cells, which then are transferred to the aforementioned transfer plate for further treatment. However, since the preparation method of the easily detachable hydrogel layer is too complicate and additional growth factors and additives are essential for the culture of spheres, it spends extremely long period (4 to 7 weeks) and high material cost in incubation.

It is therefore attempted by the applicant to deal with the above situation encountered in the prior art.

### SUMMARY OF THE INVENTION

For ease of investigating the issues on resistance to cancer therapy, invasion and metastasis, recurrence and so on, and for applying the research results in the clinical cancer therapy, the nonadhesive sphere cell culture container, system and method are researched and developed in the invention, to cultivate as the abundant sphere cells with the characteristics of CSCs in the cost-effectively and time-efficiently manner, so that the high-cost, time-consuming and additional separation issues in the prior art are overcome.

The invention provides a culture container for nonadhesive sphere cells, including: a container body having a bottom; and a gel film coated on the bottom, wherein the gel film includes an agarose gel with a concentration of 1% (w/v) to 2% (w/v).

The invention also provides a culture system for nonadhesive sphere cells, including: a container body having a bottom; a gel film coated on the bottom and including an agarose gel with a concentration of 1% (w/v) to 2% (w/v); and a liquid medium covered on the gel film, wherein the culture system is capable of cultivating cells as the sphere cells.

A culture method for nonadhesive sphere cells, including steps of coating an agarose gel with a concentration of 1% (w/v) to 2% (w/v) on a bottom of a container body to form a gel film; adding a cell and a liquid medium; and cultivating the cell as the nonadhesive sphere cells via an adequate duration.

Gel film is made by mixing agarose and liquid, heating and then solidifying. The preferred concentration of agarose gel is 1% (w/v) to 1.6% (w/v), and more preferred one is 1.2% (w/v). The above liquid is a buffer, which can be, but not limited to phosphate buffered saline (PBS), fetal bovine serum (FBS) or deionized water (ddH2O). Furthermore, the above culture container further includes a sidewall connected to the bottom, and the gel film also can be covered on the sidewall. The types of the liquid medium are selected based on the characteristics of cells.

The culture system of the invention accommodates to the various cancer cell lines, and the harvested spheres are sphere cancer cells. Cancer cells include but not limited to squamous epithelial cancer cells, adenocarcinomas, neurological carcinomas, and the sphere cancer cells relatively includes but not limited to spheric squamous epithelial cancer cells, spheric carcinomas, and spheric neurological carcinomas.

Furthermore, the above sphere cancer cells are sphere cancer stem cells. Accordingly, the above method is a method for incubating cancer stem cells. Also, the above system is a system for incubating cancer stem cells.

In concluding the above description, in the invention, a gel film is formed on the bottom of the culture container using agarose gel, to give an incubation circumstance on which cells cannot attach, so that cells form the spheres. Furthermore, cancer cells incubated using the technologies of the invention can form sphere cancer cells.

The detailed technologies and the preferred embodiments of the prevent invention are described as follows, for enabling the skilled in the art to understand the technical features of the invention accordingly.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a series of optical microscopic photographs showing human oral squamous cell carcinoma cell lines SAS and OECM-1 being cultivated as sphere cells in accordance with the present invention.

FIG. 2 depicts a diagram showing the percentage of CD133⁺ and ALDH1⁺ cells in SAS and OECM-1 parental cells and their spheres.

FIG. 3(A) depicts a bar chart showing the mRNA expression of Sox2, Oct4 and Nanog (relative to GAPDH) in SAS and OECM-1 parental cells and their spheres.

FIG. 3(B) depicts a bar chart showing the protein expression of Sox2, Oct4 and Nanog (relative to GAPDH) in SAS and OECM-1 parental cells and their spheres.

FIG. 4(A) depicts the survival (%) of SAS and OECM-1 parental cells and their spheres after radiation treatment.

FIG. 4(B) depicts the survival (%) of SAS and OECM-1 parental cells and their spheres after Cisplatin treatment.

FIG. 4(C) depicts the survival (%) of SAS and OECM-1 parental cells and their spheres after Cisplatin treatment followed by radiation treatment.

FIG. 4(D) depicts the survival (%) of SAS and OECM-1 parental cells and their spheres after radiation treatment followed by Cisplatin treatment.

FIGS. 5(A) and 5(B) respectively depict the optical microscopic photographs showing that glioma cell line GL261 is cultivated as spheres after (A) 0-day and (B) 10-days incubation periods in the invention.

FIGS. 6(A) and 6(B) respectively depict the optical microscopic photographs showing that oral cancer cell line OECM1 is cultivated as spheres after (A) 0-day and (B) 10-days incubation periods in the invention.

FIGS. 7(A) and 7(B) respectively depict the optical microscopic photographs showing that colon cancer cell line HT29 is cultivated as spheres after (A) 0-day and (B) 10-days incubation periods in the invention.

FIGS. 8(A) and 8(B) respectively depict the optical microscopic photographs showing that non-small cell lung cancer cell line NCI-H23 is cultivated as spheres after (A) 0-day and (B) 10-days incubation periods in the invention.

FIG. 9(A) depicts the optical microscopic photographs showing the spheres cultivated using agarose gel with a concentration of 2.2% (w/v).

FIG. 9(B) depicts the optical microscopic photographs showing the spheres cultivated using agarose gel with a concentration of 0.4% (w/v).

### DETAILED DESCRIPTION OF THE INVENTION

For clarity of disclosure, and not by way of limitation, the detailed description of the invention is divided into the subsections that follow.

The nonadhesive sphere cell culture container and method were exploited to cultivate sphere cells (spheres), and the method was performed by coating a layer of gel film on the bottom of the container, followed by adding the liquid medium containing a cell or cells onto the gel film until the spheres were generated via incubation.

For instance, agarose gel was added into phosphate-buffered saline (PBS), heated (or sterilized) at 80° C. to 100° C., and then this mixture was aseptically plated on the bottom of the 10-cm culture dish. As the mixture was cooled, agarose gel was solidified as a thin layer, so that the culture dish became a culture circumstance on that cells could not attach. Subsequently, 10 ml liquid medium containing cells (5×10⁴ cells/dish, supplemented with 10% fetal bovine serum) was added on the thin layer of the 10-cm culture dish, cells were incubated in the incubator with 5% CO₂ at 37° C. Replacement of medium was not essential during the incubation period. The round and smooth spheres in shape could be observed after 5-day to 7-day incubation, and the formation of the sphere stem cells could be induced with 7-day to 10-day incubation.

The liquid which can be mixed with agarose gel in the invention includes but not limit to PBS, FBS and deionized water, and the types of the mixing liquid do not have any weight on the incubation result.

To further illustrate the present invention, the following specific examples are provided.

### EMBODIMENT 1

### Incubation of Sphere Stem Cells

Embodiment 1 was performed by individually incubating two types of cancer cells to form the sphere cancer cells by using the technique of the present invention. Furthermore, in this Embodiment, the characteristics of cancer stem cells expressed on the above sphere cancer cells were determined using the method described in Experiments 1 to 4, so as to identify the above sphere cancer cells indeed were the CSCs.

The examples of the cells used in this Embodiment were human tongue cancer cell line SAS, which was incubated in Dulbecco's Modified Eagle Medium (DMEM) medium supplemented with 10% FBS, and human oral squamous carcinoma cell line OECM-1, which was incubated in Roswell Park Memorial Institute-1640 (RPMI1640) medium supplemented with 10% FBS.

### Experiment 1

### Cell Surface Markers Analyzed Using Flow Cytometry

Cells were trypsinized with trypsin-EDTA as similar to the subculture assay, washed with PBS, and resuspended (10⁶ cells in 100 μl PBS). The primary antibodies (CD133, CD24 and CD44) were respectively added, and the blank without addition of the primary antibody was the negative control. The primary antibody and cells were well mixed and incubated on ice for 1 hour. Cells were subsequently washed with PBS and centrifuged at 1,500 rpm at 4° C. for 5 minutes thrice to discard the supernatant. The corresponding secondary antibody with fluorescent labels was added, and the second antibody and cells was well mixed and incubated to avoid light on ice for 30 minutes. Subsequently, cells were washed with PBS and then centrifuged at 1,500 rpm at 4° C. for 5 minutes thrice to discard the supernatant. The total volume of cells was concentrated to 1 ml, and 10,000 cells were detected using flow cytometer. The analysis on aldehyde dehydrogenase 1 (ALDH1) was made using ALDEFLUOR® assay kit (Stemcell Technologies Inc., Vancouver, B.C., Canada). The primary antibody was individually added to the experiment, and the control to which the ALDH1 inhibitor DEAB further added, and cells were incubated on ice for one hour after cells were well mixed. Subsequently, cells were washed with PBS and centrifuged at 1,500 rpm at 4° C. for 5 minutes thrice to discard the supernatant. The total volume of cells was concentrated to 1 ml, and 10,000 cells were detected using flow cytometer.

### Experiment 2

### Reverse Transcription-Polymerase Chain Reaction (RT-PCR)

Total RNA was isolated with TRIzol® Reagent (Invitrogen, Carlsbad, Calif., U.S.A.) and the instruction manual and quantified. Reverse transcription (RT) was performed using SuperScript® III (Invitrogen) and the instruction manual. In brief, 5 μg of total RNA was reverse transcribed with SuperScript® III (Invitrogen) at 55° C. for 1 hour into the total complementary DNA (total cDNA). The PCR reaction involved an initial denaturation at 94° C. for 5 minutes, followed by annealing cDNA with the primers at 58° C. to 62° C. for 30 seconds, and followed by amplification at 72° C. for 45 seconds to obtain double strand DNA. The PCR primers for analysis of mRNA were: glyceraldehyde-3-phosphate dehydrogenase (GAPDH) sense strand (SEQ ID NO:1), GAPDH antisense strand (SEQ ID NO:2); Oct-4 (octamer-binding transcription factor 4) sense strand (SEQ ID NO:3), Oct-4 antisense strand (SEQ ID NO:4); Nanog sense strand (SEQ ID NO:5), Nanog antisense strand (SEQ ID NO:6); and Sox2 (SRY (sex determining region Y)-Box 2) sense strand (SEQ ID NO:7), Sox2 antisense strand (SEQ ID NO:8). RT-PCR products herein were run on agarose gel for electrophoresis assay recognized by the skilled person in this art.

### Experiment 3

### Western Blotting

Experiment 3 was performed after sodium dodecyl sulfate (SDS)-polyacrylamide gel electrophoresis known by the skilled person in this art, and development was made using the enhanced chemiluminescence system, and the luminescence was visualized on X-ray film. The primary antibodies were used: GAPDH (ab9482; 1:5000 dilution) (Abcam, Cambridge, Mass., U.S.A.), Oct-3/4 (sc-8630; 1:1000) (Santa Cruz Biotechnology), NANOG (sc-81961; 1:1000) and SOX2 (sc-17320; 1:500) (Santa Cruz Biotechnology) in TBST (Tris-buffered saline plus 0.1% Tween 20) buffer containing 3% nonfat milk. The secondary antibodies were used: anti-mouse secondary antibody conjugated with peroxidase (1:1000) (Santa Cruz Biotechnology) and rabbit anti-goat secondary antibody conjugated with peroxidase (1:1000) (Santa Cruz Biotechnology).

### Experiment 4

### Chemosensitivity and Radiosensitivity Assay

Cells were seeding in 10-cm cell culture dish at a density of 1×10⁶ cells/dish. For the chemosensitivity assay, cells were treated with 10 to 200 μM Cisplatin (Sigma, St Louis, Mo., U.S.A.) for 48 hours. For the radiosensitivity assay, cells were irradiated using a CyberKnife® radiosurgery system (Accuray, Sunnyvale, Calif., U.S.A.) to deliver different doses (2-10 Gy). Relative survival fraction of cells was determined by MTS assay using the CellTiter® 96 Aqueous One Solution Cell Proliferation Assay kit (Promega, Madison, Wis., U.S.A.) after 36 hours of radiation treatment.

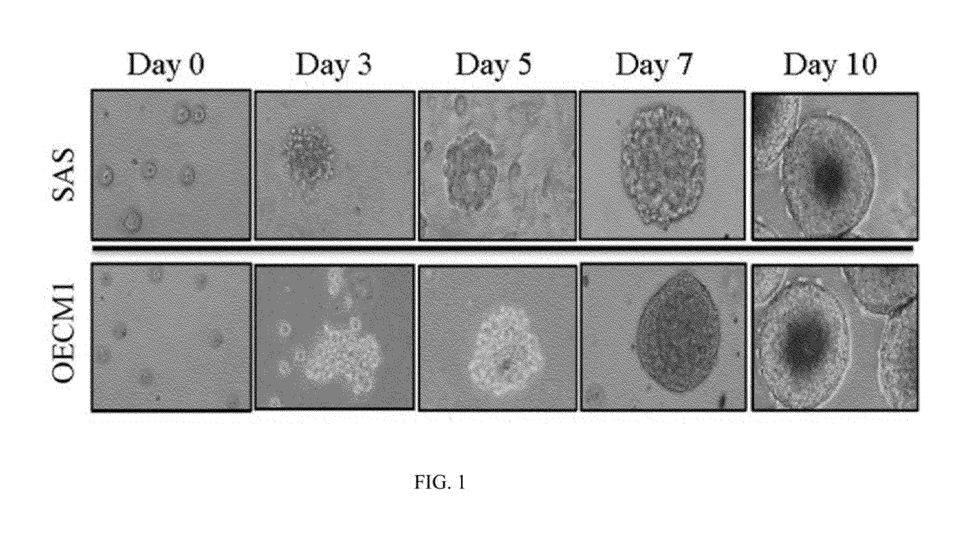

Please refer to FIG. 1, which depicts a series of optical microscopic photographs showing that SAS and OECM-1 cells were respectively induced as spheres using the nonadhesive sphere cell culture system of the invention. From a series of photographs of SAS cells (the upper row) and OECM-1 cells (the lower row), it could be known that the suspending cancer cells gradually differentiated into the three-dimensional balls with a spheroid configuration as the cultivation period increases. The larger floating spheres formed when cells were incubated on Days 3 to 5, and the spheres with a round and smooth contour formed when cells were continuously incubated on Days 5 to 7. Incubation went on and the number of spheres gradually increased and the volume of each sphere was enlarged. These sphere cells appeared a tightly attached and complete 3D configuration which was not easily disrupted.

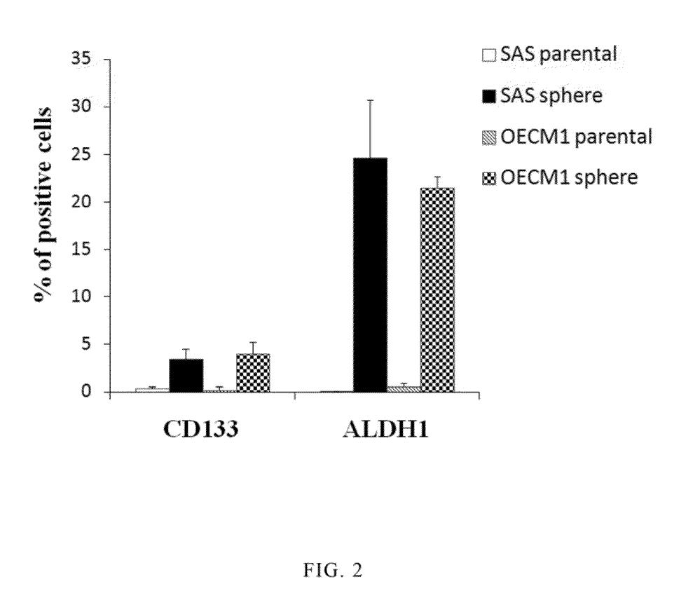

Please refer to FIG. 2, which depicts a diagram showing the percentage of CD133⁺ and ALDH1⁺ cells in SAS and OECM-1 parental cells and their spheres. FIG. 2 was afforded and plotted in accordance with the quantification calculation from the results of flow cytometry, and the qualification was well known by the skilled person in this art and was not described herein. In FIG. 2, expression of CD133 and ALDH1 was usually absent or very low in SAS and OECM-1 parental cells, whereas a 3% to 4% increase in CD133 expression and a 20% to 30% increase in ALDH1 expression in the spheres compared with the parental cells. That is, the levels of expression of CD133 and ALDH1 were significantly higher in the spheres than they were in the parental cells.

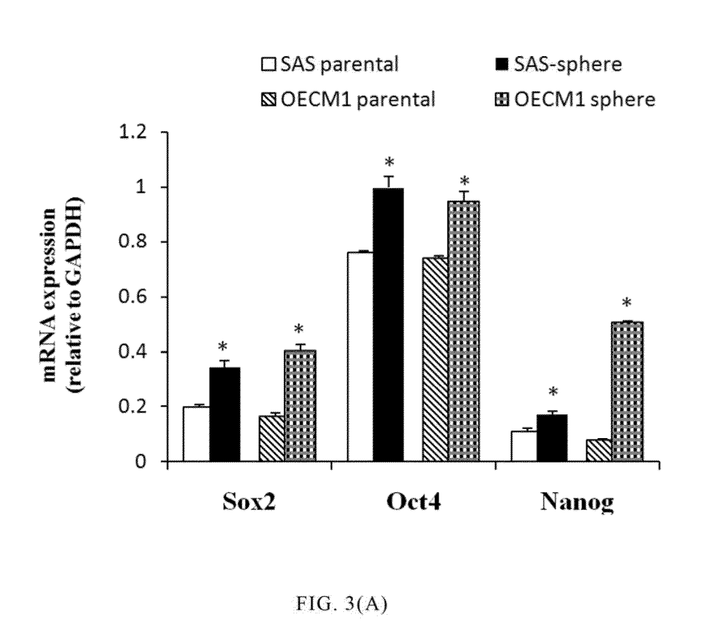

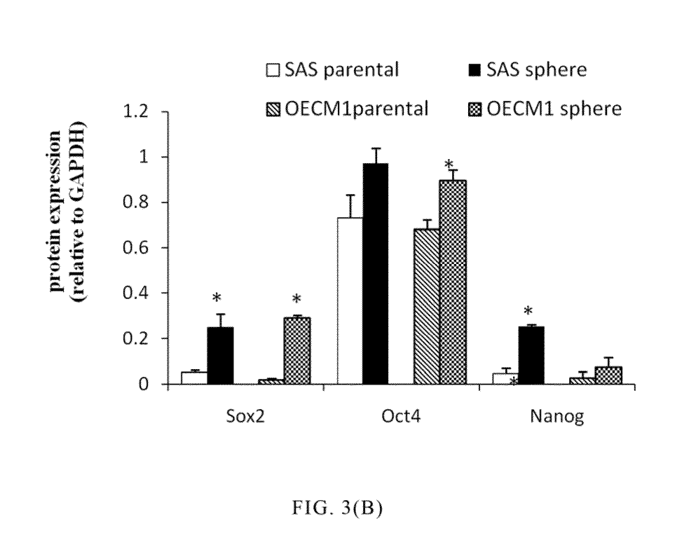

Please refer to FIG. 3(A), which depicts a bar chart showing the mRNA expression of Sox2, Oct4 and Nanog (relative to GAPDH) in SAS and OECM-1 parental cells and their spheres. These results were obtained using the experimental data of RT-PCR. The levels of Sox2, Oct4, and NANOG transcripts were significantly increased in SAS and OECM-1 spheres compared with their parental cells. Please refer to FIG. 3(B), which depicts a bar chart showing the protein expression of Sox2, Oct4 and Nanog (relative to GAPDH) in SAS and OECM-1 parental cells and their spheres. The level of protein expression was obtained by calculating the experiments result of Western blotting. It was revealed that the expression of the Sox2, Oct4, and Nanog proteins was upregulated in SAS and OECM-1 spheres compared with their parental cells. The experimental results of the immunofluorescence staining also revealed that the cellular levels of CD133, ALDH1, Sox2, Oct4 and Nanog in the spheres increased (data not shown).

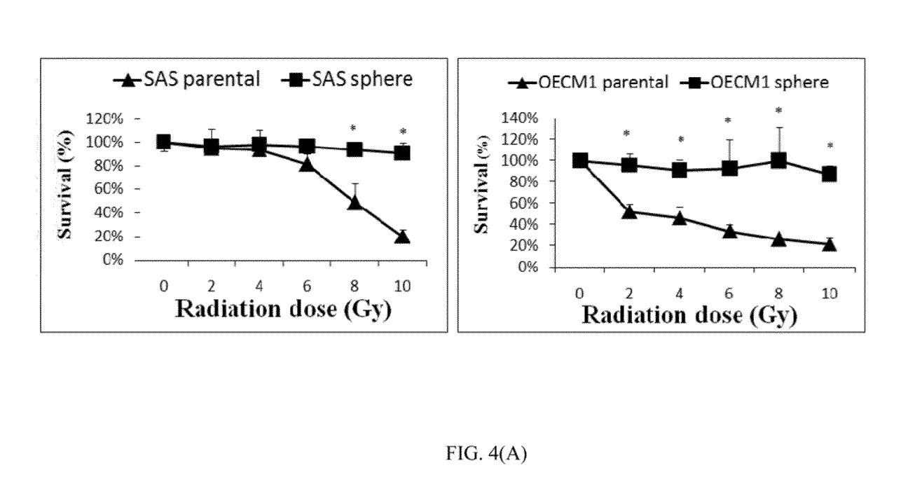

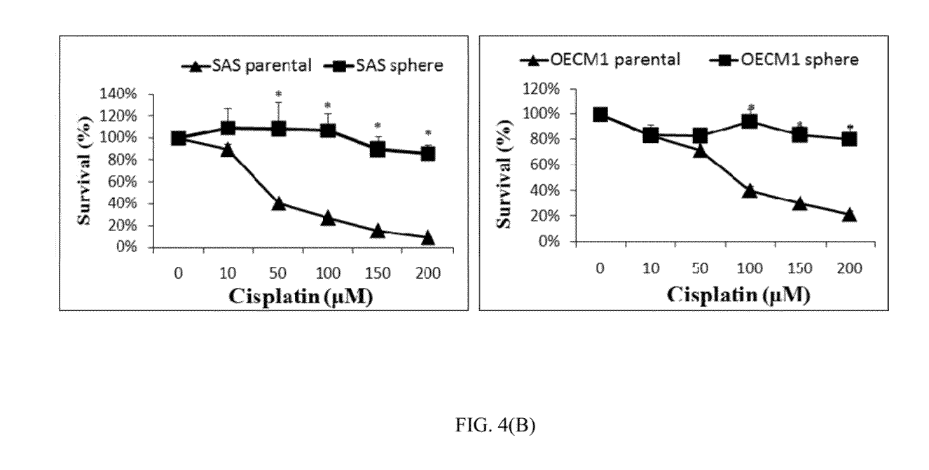

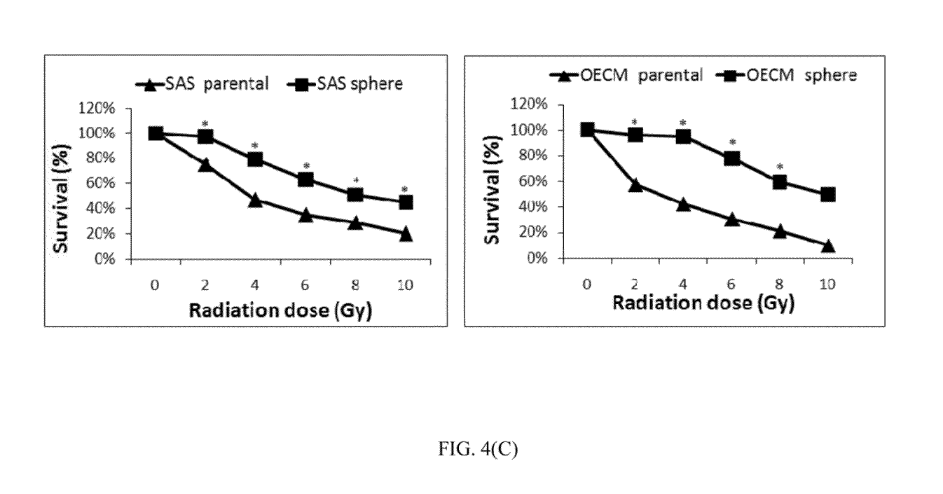

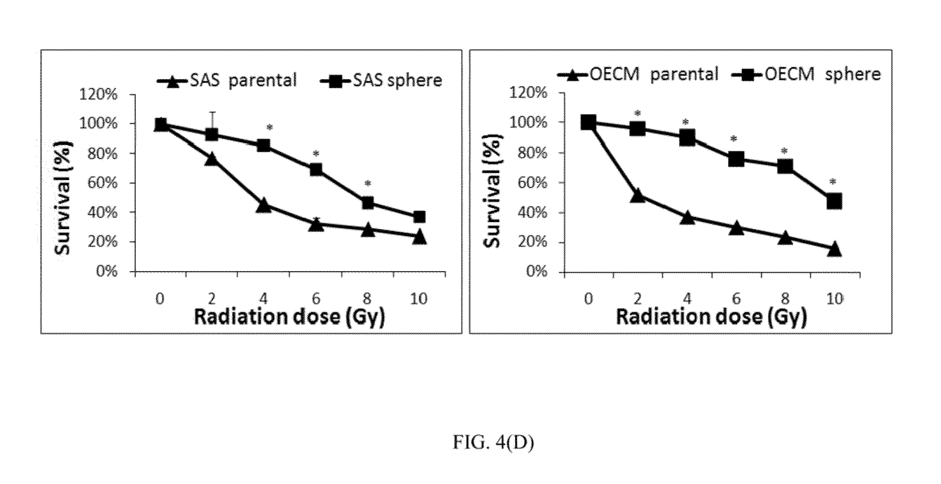

Please refer to FIG. 4(A), which depicts the survival (%) of SAS and OECM-1 parental cells and their spheres after radiation treatment, and cell viability was evaluated after these cells were treated with radiation doses up to 10 Gy for 36 hours and were subject to a MTS assay. In FIG. 4(A), SAS and OECM-1 spheres were more radioresistant than their parental cells. Please refer to FIG. 4(B), which depicts the survival (%) of SAS and OECM-1 parental cells and their spheres after the Cisplatin treatment, and cell viability was evaluated after these cells were treated with Cisplatin doses up to 200 μM for 48 hours and were subject to a MTS (3-(4,5-dimethylthiazol-2-yl)-5-(3-carboxymethoxyphenyl)-2-(4-sulfophenyl)-2H-tetrazolium) assay. In FIG. 4(B), SAS and OECM-1 spheres were more resistant to Cisplatin than their parental cells.

To imitate the clinical condition, a combined chemo- and radiotherapy (CCRT) treatment was administered on SAS and OECM-1 parental cells and their spheres.

Please refer to FIG. 4(C), which depicts the survival (%) of SAS and OECM-1 parental cells and their spheres after Cisplatin treatment followed by radiation treatment. This assay was performed by treating cells with chemotherapy using 20 μM Cisplatin for 24 hours followed by radiotherapy. In FIG. 4(C), the combinations were more effective in reducing the survival rate of the parental cells and spheres compared with the single treatment, and SAS and OECM-1 spheres were more resistant than the parental cells (with variable significance levels) when using the combined treatment.

Please refer to FIG. 4(D), which depicts the survival (%) of SAS and OECM-1 parental cells and their spheres after radiation treatment followed by Cisplatin treatment. This assay was performed by treating cells with radiotherapy followed by chemotherapy using 20 μM Cisplatin for 24 hours, and the consistent conclusion also was obtained with that in FIG. 4(C). It could be known from FIGS. 4(C) and 4(D) that the aforementioned combined therapies could be effective in the treatment of spheres and their parental cells, and were not limited to the order of the chemotherapy and radiotherapy.

In addition, the inventors not only demonstrated sphere cancer cells were the cancer stem cells using Experiments 1 to 4, but also proved that the sphere cancer cells had the characteristics of cancer stem cells via immunohistochemical (IHC) staining, immunofluorescence cell staining, tumor cells xenograft assay in the subcutaneous tissue of mice, migration and invasion assays (data not shown).

Therefore, Embodiment 1 of the invention was demonstrated that cells could be incubated as sphere cells, and cancer cells further could be incubated as sphere cancer stem cells using the technologies of the invention.

### EMBODIMENT 2

### Incubation of Spheres from Other Cancer Cells

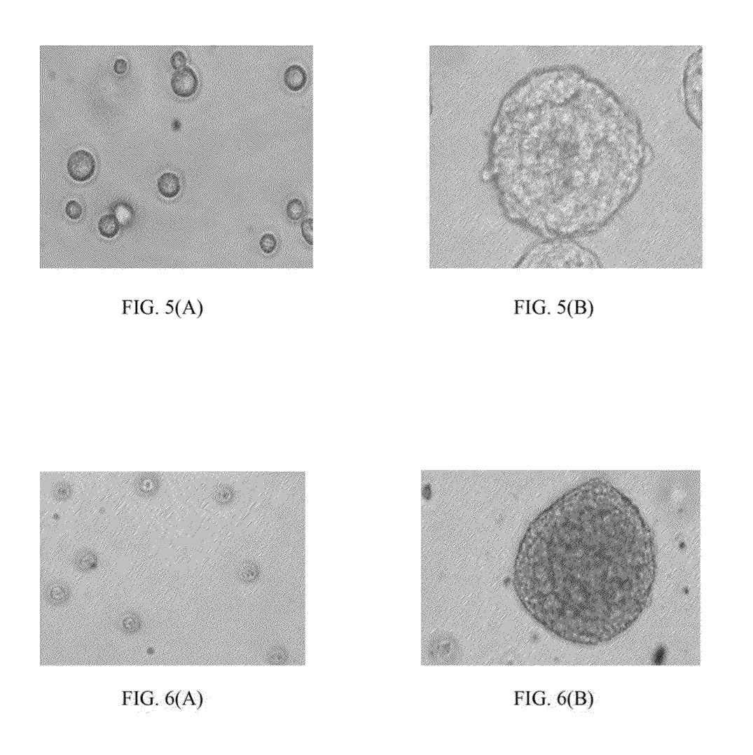

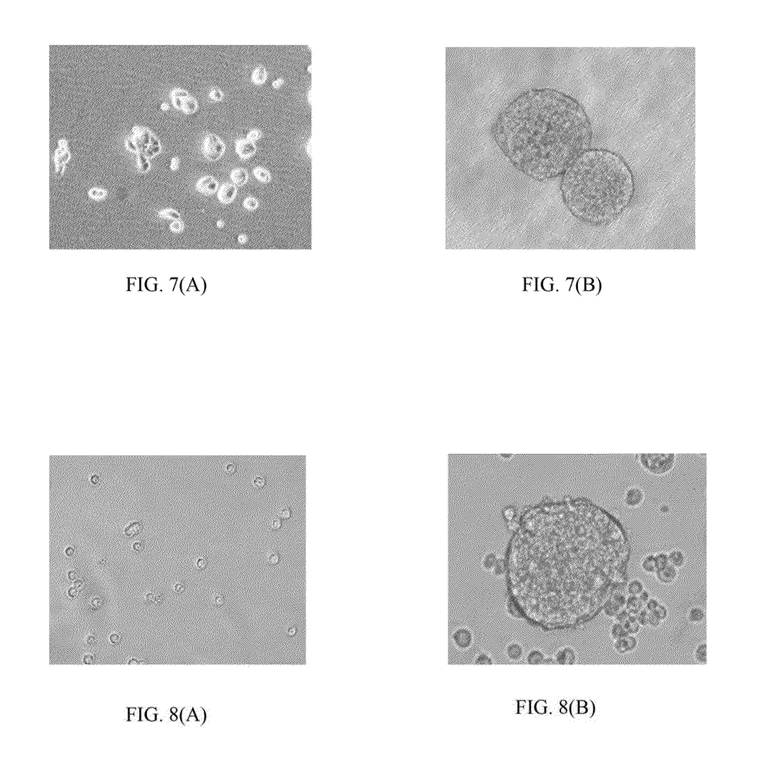

Using the technologies of the invention, four different types of cancer cell lines were incubated as sphere cancer cells in Embodiment 2. Please refer to FIGS. 5(A), 5(B), 6(A), 6(B), 7(A), 7(B), 8(A) and 8(B), the cultured cancer cell lines were glioma cell line GL261 (FIGS. 5(A) and 5(B)), oral cancer cell line OECM1 (FIGS. 6(A) and 6(B)), colon cancer cell line HT29 (FIGS. 7(A) and 7(B)), and non-small cell lung cancer cell line NCI-H23 (FIGS. 8(A) and 8(B)), respectively. Cells revealed as loose and singular cell on Day 0 (referring to FIGS. 5(A), 6(A), 7(A) and 8(A)), and cells forms the sphere cells with a round and smooth contour configuration after 10-days incubation period (referring to FIGS. 5(B), 6(B), 7(B) and 8(B)). Accordingly, the technologies of the invention may be applied in the various cell lines, which are not limited as described herein.

The volume of the used agarose gel for forming the gel film in the culture dish depends on the types and the base areas of culture container, and the volume of agarose gel needed in reference for the various culture containers is shown in Table 1.

TABLE 1

| | Flask | | Dish | | Culture Plate | | |
|---|---|---|---|---|---|---|---|
| | T75 | T25 | 15 cm | 10 cm | 6 well | 12 well | 24 well |
| Volume of | 6 | 3 | 12 | 5 | 1.5 | 1.0 | 0.5 |
| agarose | | | | | | | |
| gel (ml) | | | | | | | |

The concentration of agarose gel adequate for the invention depends on the types of the desired cell lines. In general, the concentration of agarose gel was lowered than 2% (w/v), or was ranged between 1% (w/v) and 1.6% (w/v), or was 1.2% (w/v).

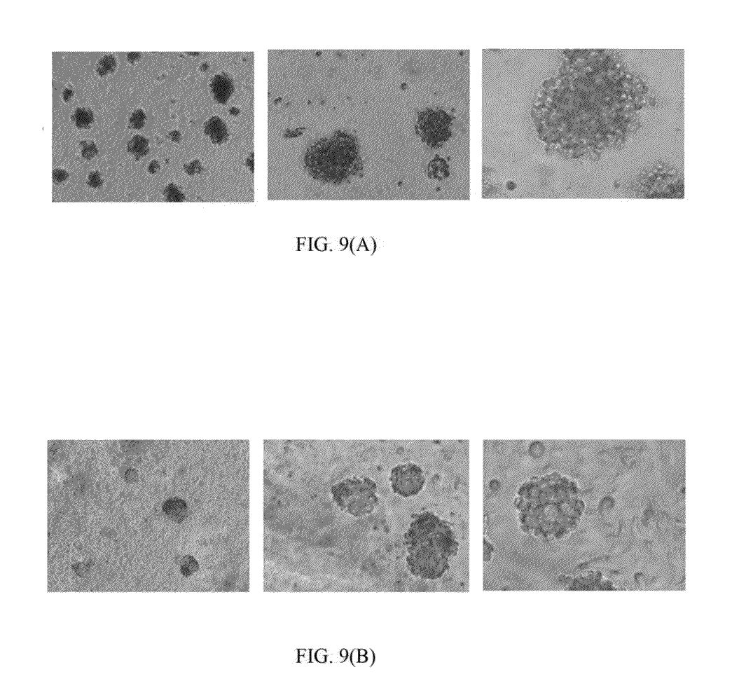

It should be comprehended that although cells would form as the spheroid configuration at a slightly higher concentration of agarose gel (2.2% (w/v), for example), most sphere cells would be embedded into the gel film made of agarose gel. Thus, too high concentration of agarose gel is unfavorable in harvesting spheres (referring to FIG. 9(A)). In addition, such sphere cells did not reveal the complete, round, and smooth spheroid configuration but a botryoidal aggregation, and had an un-complete 3D configuration and differentiated floatingly. Please, also refer to FIG. 9(B), most cells would be attached on the gel film to grow and differentiate at a more lower concentration of agarose gel (0.4% (w/v), for example) since it could not cause a circumstance for floating growth and differentiation.

In conclusion, a layer of gel film is formed on the bottom of the culture container using agarose gel in the invention, so that an incubation circumstance on which cells cannot adhere/attach to cause cells to grow and differentiate into sphere cells via incubation. Furthermore, cancer cells can be incubated as sphere cancer cells using the technologies of the invention, and it has been demonstrated that the sphere cancer cells incubated using the technologies thereof have the characteristics of CSCs. Accordingly, the invention is beneficial in effectively reducing cost and efficiently saving incubation period, as well as simultaneously incubating an abundant amount of CSCs for researches on tumor initiation, cancer progression, invasion and metastasis of cancer cells, cancer therapy resistance, and recurrence.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A culture container for nonadhesive sphere cells, comprising:
a container body having a bottom; and
a gel film coated on the bottom,
wherein the gel film is an agarose gel with a concentration of 1% (w/v) to 2% (w/v).

2. The culture container according to claim 1, wherein the culture container further comprises a liquid medium covered on the gel film.

3. The culture container according to claim 2, wherein the liquid medium further comprises a cell.

4. The culture container according to claim 1, wherein the container body further comprises a sidewall connected to the bottom, and the gel film further is coated on the sidewall.

5. A culture system for nonadhesive sphere cells, comprising:
a container body having a bottom;
a gel film coated on the bottom and the gel film is an agarose gel with a concentration of 1% (w/v) to 2% (w/v); and
a liquid medium covered on the gel film,
wherein the culture system is capable of cultivating cells as the sphere cells.

6. The culture system according to claim 5, wherein the concentration of the agarose gel is ranged between 1% (w/v) and 1.6% (w/v).

7. The culture system according to claim 5, wherein the concentration of the agarose gel is 1.2% (w/v).

8. The culture system according to claim 5, wherein the liquid medium is capable of selected based on the characteristics of the cells.

9. The culture system according to claim 5, wherein the cell is a cancer cell, and the sphere cells are sphere cancer cells.

10. The culture system according to claim 9, wherein the sphere cancer cells are sphere cancer stem cells

11. The culture system according to claim 10, wherein the system is a system capable of cultivating cancer stem cells.

12. A culture method for nonadhesive sphere cells, comprising steps of:
coating an agarose gel with a concentration of 1% (w/v) to 2% (w/v) on a bottom of a container body to form a gel film;
adding a cell and a liquid medium; and
cultivating the cell as the nonadhesive sphere cells via an adequate duration.

13. The culture method according to claim 12, wherein the gel film is capable of made by mixing the agarose gel with a liquid, heating to melt and then solidifying the agarose gel.

14. The culture method according to claim 13, wherein the liquid is selected from the group consisting of phosphate-buffered saline, bovine serum albumin and deionized water.

15. The culture method according to claim 12, wherein the cell is a cancer cell, and the sphere cells are sphere cancer cells.

16. The culture method according to claim 15, wherein the cancer cell is selected from squamous epithelial cancer cell, adenocarcinoma and the neurological carcinoma.

17. The culture method according to claim 15, wherein the sphere cancer cells are sphere cancer stem cells.

18. The culture method according to claim 17, wherein the method is a method capable of cultivating cancer stem cells.

19. The method according to claim 12, wherein the adequate duration is 5 days to 12 days.

20. The method according to claim 12, wherein the adequate duration is 7 days to 10 days.
